(19) United States
(12) Patent Application Publication
(10) Pub. No.: US 2014/0274650 A1
(43) Pub. Date: Sep. 18, 2014

(54) SEGREGATION OF OILS IN THE FRACTIONATION OF ASPIRATED ADIPOSE TISSUES

(71) Applicant: James R. Ellsworth, Marshfield, MA (US)

(72) Inventor: James R. Ellsworth, Marshfield, MA (US)

(73) Assignee: HARVEST TECHNOLOGIES CORPORATION, Plymouth, MA (US)

(21) Appl. No.: 14/239,274

(22) PCT Filed: Aug. 16, 2012
(86) PCT No.: PCT/US2012/051070

§ 371 Date: May 2, 2014

Related U.S. Application Data
(60) Provisional application No. 61/524,345, filed on Aug. 17, 2011.

(51) Int. Cl. A61L 27/24

(52) U.S. Cl. CPC A61L 27/24
USPC 494/37; 422/533

(57) ABSTRACT

Adipose is obtained from aspirated adipose by centrifugal separation of adipose from tumescent fluids and oils released from damaged adipose cells. Aspirated adipose is placed in a container having therein a disk that floats on adipose and takes up the released oils whereby they are unlikely to remix with the adipose during handling after centrifugation. The disk also adheres to the adipose by entraining part of the adipose layer.

### TECHNICAL FIELD

This invention relates to the separation of adipose tissue from aspirated tissues. In a preferred embodiment, the separation is by centrifugation.

### BACKGROUND ART

The aspiration of adipose tissue, also known as liposuction, is known in cosmetic surgery. Small volumes of less than about 20 ml are routinely injected, for example, into the lips to alter the patient's appearance. More recently adipose tissue has been used in the field of regenerative injection therapy. Small amounts of adipose tissue can be injected into defects to fill them and to act as a scaffold for soft-tissue repair.

Adipose tissues are aspirated by first injecting a tumescent fluid into the area from which the adipose tissue is to be removed. The practitioner will then aspirate adipose tissue by inserting a needle or cannula into the area where the tumescent fluid has been injected and applying a vacuum. The fat and tumescent fluids are then typically allowed to partially fractionate by standing in a tube or syringe whereby the fractions of different densities separate by gravity. It has also been found useful in subsequent handling and to improve the quality of the adipose tissue to process the aspirated fluid in a centrifuge at greater than 1 G force, but less than 2000G. The centrifugal forces separate the tumescent fluid from the adipose tissue, and oil from damaged adipose cells separates as a third, least-dense fraction above the adipose tissue.

The tumescent fluid can be expressed from a container, such as a tube or syringe after being subjected to centrifugation as a first step. A problem, however, is that the oil remains in the syringe above the adipose tissue and can remix with the adipose tissue as the syringe is handled.

### SUMMARY OF THE INVENTION

In accordance with the invention, a container having the aspirated fluids therein is placed in a centrifuge to separate the adipose tissue from the tumescent fluid according to their densities. The container is provided with an element designed to float above the layer of adipose tissue after centrifugal separation. The floating element is made of a material that will absorb the oils that separate from the aspirated fluid during centrifugation and at least partially retain them in the element to prevent their remixing with the adipose tissue during handling of the container and removal of the separated fluids from the container. In a preferred embodiment, the container is a syringe that is also used initially to aspirate the fluids from the patient. Alternatively, the container is a syringe to which the aspirated fluids are transferred after aspiration or any another container that receives the aspirated fluids and is capable of being subjected to centrifugal forces.

Preferably, the oil-absorbing element is made of a material having a density such that it automatically positions itself between the adipose-cells fraction and the less-dense oil fraction after centrifugation. The floating element may have a density between about 0.905 to about 0.925.

It is also preferred that the floating element be porous such that during centrifugation, as the less dense fraction of oil is forming, the oil is entrained in the floating element. Mixing of the oil back into the adipose during post-centrifugation handling by a practitioner is prevented, because the forces typically applied during handling are too small to cause escape of an undesirable portion of the entrained oil from the porous floating element. In one preferred embodiment, the floating element is made of a porous plastic sold under the trademark POREX with pore sizes between about 20 and about 170 microns and more preferably between about 90 and about 130 microns.

The floating element can, however, be made of other materials and can be solid as well as porous. One advantage of the preferred porous material is that it also entrains some of the adipose, and in the specific embodiments described as much as about 20% of the thickness of the floating element may be in the adipose layer itself. This tends to attach the floating element to the adipose layer and ensure that the floating element remains between the oil and adipose layers to prevent mixing of these layers during handling subsequent to centrifugation.

It is also contemplated that the floating element be made of a combination of solid and porous layers to separate the oil and adipose layers and also entrain them to achieve the advantages noted. For example a floating element may be made of a solid material on the upper part and a porous layer on the bottom. The porous layer may entrain more or less oil as desired by making it thicker or thinner.

Additionally, materials other than the preferred porous material are capable of entraining, or even absorbing, the oil layer, and other materials that attach to the adipose layer may be used. Thus, while porous materials are preferred for the floating element, other materials that are attracted to the adipose layer and entrain or absorb the oil layer or prevent mixing of the oil layer with the adipose can be used.

In a preferred embodiment adipose tissues are aspirated from the patient with a syringe having a handle that can be detached to allow the syringe to be placed in a centrifuge. In this embodiment, the syringe handle is attached to the plunger that carries a seal such that the handle can be removed to allow centrifugation of the syringe and fluids, after which the most-dense fraction, the mixture of tumescent fluid and water, is expelled from the syringe by reattaching the handle and pushing on the plunger. The adipose tissues can then be introduced to the patient by further pushing on the plunger or by removing them from the syringe in known manner, such as by using another syringe or a vacuum pump. The oils are retained in the floating element notwithstanding the pressure on the plunger and do not mix with the adipose tissues or otherwise present a problem.

The syringe and floating element with the oils retained therein can be disposed after removal of the adipose tissues.

### BRIEF DESCRIPTION OF THE DRAWINGS

FIG. 1 is a side view of a prior art syringe with aspirated adipose fluids before density fractionation.

FIG. 2 is a side view of a prior art syringe with aspirated adipose fluids after density fractionation.

FIG. 3 is a side view of a syringe in accordance with the invention having aspirated adipose fluids before density fractionation.

FIG. 4 is a side view of a syringe in accordance with the invention with aspirated adipose fluids after density fractionation.

### DETAILED DESCRIPTION OF THE INVENTION

With reference to FIG. 1, a container for aspirated adipose fluids is illustrated. In a preferred embodiment the container is a syringe 2 with a syringe barrel 4 that forms a cavity for receiving aspirated fluids 6. The aspirated fluids comprise adipose cells, tumescent fluid, and oils from damaged (ruptured) adipose cells. The syringe includes a plunger that moves within the barrel and comprises a carrier 8 and a seal 10. The carrier includes a handle connector 12 for engaging a removable handle (not illustrated). When a handle is attached, a user can move the plunger in the barrel by manipulating the handle. The handle can be removed to allow placement of the syringe in a centrifuge and then reattached after centrifugation to express the fractionated fluids.

The syringe may also include a cap 14, which seals the end of the syringe against leakage during centrifugation. The fluid port end of the syringe may have any of a variety of connectors, such as a Luer-type connector, to receive a needle, cannula, tube, or the like, and the cap 14 is configured to engage the particular type of connector.

FIG. 2 illustrates the syringe of FIG. 1 after density fractionation of the aspirated fluid by centrifugation. The fractionation illustrated in FIG. 2 results generally in a first layer 16, which is the most dense and comprises tumescent fluid, a second layer 18, which comprises adipose tissues, and a third layer 20, which is least dense and comprises oils released by rupture of adipose cells. It will be appreciated that other layers (or sub-layers) might form as well.

A problem presented by the syringe illustrated in FIG. 2 is that the oil layer 20 can remix with the other layers, particularly the adipose layer 18, by handling the syringe during its removal from the centrifuge or during manipulation of the plunger during expression of the layers of tumescent fluid and adipose tissues from the syringe. It is advantageous therefore to prevent remixing of the oil layer with the adipose fluids to avoid contamination of the adipose fluids.

FIG. 3 illustrates a syringe in accordance with the invention, wherein an element 22 is provided in the syringe 2 to preserve the segregation of the oil layer 20 that occurs during the centrifugation. The density of the material from which element 22 is made is preferably such that before centrifugation it will float on or slightly within the aspirated fluid 6. As centrifugation proceeds, element 22 takes up the oils as they separate from the fluid 6, for example, by absorption or adsorption. The density of the element 22 is chosen such that it floats at an upper portion of the adipose layer 18 after accumulation of the oils. FIG. 4 illustrates the situation after centrifugation where the oils 20, which have separated from the aspirated fluids 6 by the forces of centrifugation, have been accumulated by the floating element 22. The floating element thus segregates the oils in a safe location above the adipose tissues 18.

Element 22 may be in the shape of a disk or a variety of other shapes. In a preferred embodiment, the density of element 22 is between those of the oils 20 and the adipose 18. In this embodiment, the element 22 floats in an upper portion of the adipose layer and the material (preferably porous) entrains some of the adipose. This improves handling by establishing a physical connection between the element 22 and the adipose, which provides more stability. For example, if the syringe is laid on its side after centrifugation, the attachment of element 22 to the adipose makes it less likely that it will float in the oil layer away from the adipose and allow some remixing.

In a preferred embodiment, element 22 is in the shape of a disk having a height of about one-quarter inch, and about twenty percent of that height is in the adipose after centrifugation. The remaining part of the disk is in the oil layer 20.

While FIG. 4 illustrates a situation where all of the oils have been taken up by element 22, it is not a requirement that element 22 be large enough to take up all of the oil. In a more general case, the amount of oil is larger than can be taken up by element 22, and a layer of oil forms above the element 22. Element 22 nevertheless prevents mixing the oil into the adipose layer because the element 22 forms a barrier between the oil and the adipose cells.

In use, a user attaches a handle to the carrier 12 and a needle to syringe 2. The needle is inserted into an area from which adipose tissues are to be drawn, which has typically previously been treated with tumescent fluids, for example, to anesthetize the area. Fluids containing the target adipose cells are aspirated into the syringe by pulling on the handle, and this also draws some of the tumescent fluids into the syringe. The handle can then be removed and the syringe placed in a centrifuge for centrifugal separation of the adipose tissues from the tumescent fluids, after which the handle is reattached and the separated fluids expressed from the syringe. The tumescent fluids are the first to be expressed followed by the adipose tissues. As discussed above, some of the adipose tissues will be damaged in the aspiration and subsequent processing, which will release some oil. This oil separates as a third layer above the adipose tissues. Because the element 22 prevents remixing between the oil and the adipose tissues, the recovered adipose tissues expressed from the syringe are more pure.

Modifications within the scope of the appended claims will be apparent to those of skill in the art.

I claim:

1. A container forming a cavity for separating aspirated adipose fluids into components based on relative densities and having therein a disk movable within said cavity, said disk being made of a material and configured such that it floats on a layer of adipose that has been separated from oils released from damaged adipose cells and prevents substantially remixing of said oils with said adipose during handling of said container.

2. A container according to claim 1 wherein said material adheres to at least a portion of said layer of adipose.

3. A container according to claim 1 wherein said material is capable of taking up oils released from damaged adipose cells.

4. A container according to claim 3 wherein said material adheres to at least a portion of said layer of adipose.

5. A container according to claim 4 wherein said material is porous and the diameters of said pores are from 20 to 170 microns.

6. A container according to claim 5 wherein the diameters of said pores are from 90 to 130 microns.

7. A container according to claim 5 wherein said material is plastic.

8. A container according to claim 6 wherein said plastic is polyethylene.

9. A container according to claim 5 wherein the density of said material is between about 0.905 and 0.925.

10. A container according to claim 1 wherein said container comprises an inlet at one end for introducing said aspirated adipose fluids into said cavity.

11. A container according to claim 1 wherein said container further comprises a piston movable along said cavity for drawing said aspirated adipose fluids into said cavity, and a handle removably attached to said piston for operating said piston.

12. A method of providing adipose comprising the steps of placing aspirated adipose into a container having a cavity for separating aspirated adipose fluids into components based on relative densities and having therein a disk movable within said cavity, said disk being made of materials and configured such that it floats on a layer of adipose that has been separated from oils released from damaged adipose cells and prevents substantially remixing of said oils with said adipose during handling of said container, and subjecting said container with aspirated adipose therein to centrifugal forces to separate said aspirated adipose into at least a layer of adipose and a layer of oil released from damaged adipose cells.
